(19) United States
(12) Patent Application Publication
(10) Pub. No.: US 2014/0066799 A1
(43) Pub. Date: Mar. 6, 2014

(54) SCHEMA FOR USING DYNAMIC COLOR AND PATTERN BACKGROUNDS FOR ELECTROCARDIOGRAM DISPLAYS AND ASSOCIATED SYSTEMS AND METHODS

(71) Applicant: UNIVERSITY OF WASHINGTON through its Center for Commercialization, Seattle, WA (US)

(72) Inventors: Lawrence D. Sherman, Bellevue, WA (US); Elijah D. Christensen, Port Orchard, WA (US); Mickey Eisenberg, Seattle, WA (US); Peter J. Kudenchuk, Normandy Park, WA (US); Christopher Neils, Lake Forest Park, WA (US); Thomas Rea, Seattle, WA (US)

(21) Appl. No.: 14/018,384

(22) Filed: Sep. 4, 2013

Related U.S. Application Data
(60) Provisional application No. 61/696,663, filed on Sep. 4, 2012.

(51) Int. Cl. A61B 5/044

(52) U.S. Cl. CPC A61B 5/044
USPC 600/518; 600/523

(57) ABSTRACT

The present technology is generally directed to schemas for using dynamic color and pattern backgrounds for electrocardiogram display and associated systems and methods. In a particular embodiment, a method of displaying an electrocardiogram rhythm can include receiving an electrocardiogram signal and applying an algorithm to the signal to determine a rhythm diagnosis or recommended therapy. The method can further include displaying a color and/or a pattern corresponding to the rhythm diagnosis or recommended therapy. In various embodiments, the method can be performed in real time or on a pre-recorded signal for post-event analysis.

### CROSS-REFERENCE TO RELATED APPLICATION

This application claims the benefit of U.S. Provisional Application No. 61/696,663, entitled “SCHEMA FOR COLOR CODING REAL TIME ECG WAVEFORMS AND ITS IMPLEMENTATION”, filed Sep. 4, 2012, which is incorporated by reference herein in its entirety. Further, components and features of embodiments disclosed in the application incorporated by reference may be combined with various components and features disclosed and claimed in the present application.

### TECHNICAL FIELD

The present technology is generally directed to schemas for using dynamic color and pattern backgrounds for electrocardiogram displays and associated systems and methods.

### BACKGROUND

An electrocardiogram (ECG) is a recording of an interpretation of the electrical activity of the heart over a period of time, as detected by electrodes attached to the surface of the skin and recorded by a device external to the body. An ECG is used to measure the rate and regularity of heartbeats (commonly referred to as heart rhythm), and any abnormality in the heart rhythm, as well as the presence of any damage to the heart, and the effects of drugs or devices used to regulate the heart, such as a pacemaker. ECGs are widely used as a diagnostic tool in the clinical setting.

During continuous rhythm monitoring, an ECG signal is displayed as a continuous line trace, with time represented on the x-axis and voltage represented on the y-axis. The signal is traditionally plotted against a grid background, with grid marks indicating voltage and time increments. In other cases, the ECG signal is displayed against a blank background. In several situations, the ECG signal can be obscured by artifacts or noise and is thus unusable to a clinician. When cardiac rhythm is of primary concern, an automated computer analysis of the ECG may be used to characterize the rhythm. A conventional approach is to present the rhythm diagnosis as a text display to the clinician. However, when cardiac rhythm is being acquired as a continuous real-time display, the use of text can be cumbersome. Changes in rhythm may become less obvious to the clinician who must read each changing text field, some of which may not arouse the clinician's immediate attention. This can be potentially life-threatening, particularly if a dangerous change in rhythm is not quickly identified, increasing the risk of a negative patient outcome in critical-care environments or other emergent situations. In addition, if the ECG itself is obscured by artifact, communicating the rhythm state with text alone loses the dynamic (time-course) information that might be identified in a readable ECG signal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a partially schematic illustration of an ECG system configured in accordance with embodiments of the technology.

FIG. 2 is an illustration of an ECG trace overlaying a dynamic background and configured in accordance with embodiments of the technology.

FIG. 3 is an illustration of an ECG trace overlaying a dynamic background and configured in accordance with further embodiments of the technology.

FIG. 4 is an illustration of an ECG trace overlaying a dynamic background and configured in accordance with still further embodiments of the technology.

FIG. 5 is an illustration of a case review of a rhythm diagnosis as represented by a dynamic color schema and configured in accordance with embodiments of the technology.

FIG. 6 is a block diagram illustrating a method of displaying an ECG rhythm in accordance with embodiments of the technology.

FIG. 7 is a block diagram illustrating a method of conveying information about an ECG rhythm in accordance with embodiments of the technology.

### DETAILED DESCRIPTION

The present technology is generally directed to schemas for using dynamic color and pattern backgrounds for ECG displays and associated systems and methods. In a particular embodiment, a method of displaying an ECG rhythm can include receiving an ECG signal and applying an algorithm to the signal to determine a rhythm diagnosis or recommended therapy. The method can further include displaying a color or pattern corresponding to the rhythm diagnosis or recommended therapy. In various embodiments, the method can be performed in real time or on a pre-recorded signal for post-event analysis.

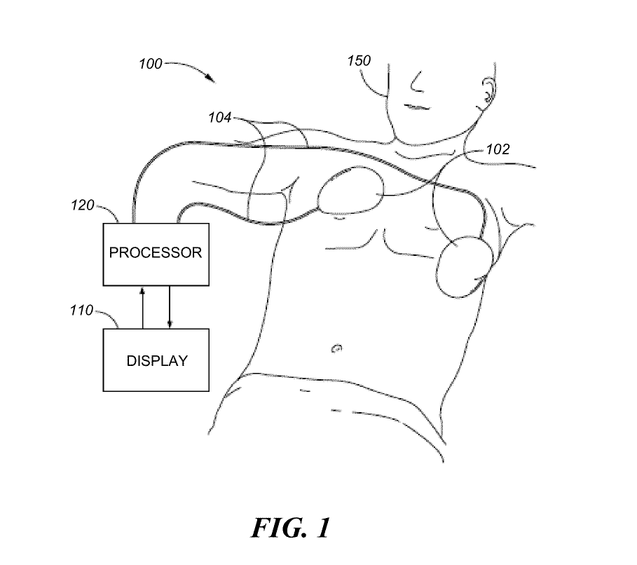

Specific details of several embodiments of the technology are described below with reference to FIGS. 1-7. Other details describing well-known structures and systems often associated with ECGs and display devices have not been set forth in the following disclosure to avoid unnecessarily obscuring the description of the various embodiments of the technology. Many of the details, dimensions, angles, and other features shown in the Figures are merely illustrative of particular embodiments of the technology. Accordingly, other embodiments can have other details, dimensions, angles, and/or features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 1-7.

FIG. 1 is a partially schematic illustration of an ECG system 100 (“the system 100”) configured in accordance with embodiments of the technology. The system 100 includes a computing device (e.g., a processor 120) coupled to a patient 150 by means of two or more electrodes 102 coupled to the processor 120 with wires 104. The electrodes 102 may be monitoring a voltage signal, impedance, and/or other values. The processor 120 can carry the signal (i.e., time-series ECG voltages) as well as other diagnostic or therapy information to a cardiac display 110. The display 110 can be an ECG monitor, automatic external defibrillator (AED), or presentation of the ECG on any other screen or display device. In further embodiments, the display 110 and processor 120 may comprise a single, unitary device, or may comprise more than two devices.

The processor 120 can be part of any embedded circuitry, personal computer, tablet, mobile computing device, or other physical computer-readable storage medium, either stand-alone or as part of a larger system such as an integrated life support system or defibrillator. The processor 120 can implement instructions which comprise receiving an ECG signal and determining a rhythm diagnosis or recommended therapy for the signal. In some embodiments, for example, the processor 120 can analyze the time series voltages of an ECG as derived from the patient 150 and determine if the rhythm is shockable or non-shockable. Based on the results of the ECG signal analysis, the processor 120 can direct the display 110 to alter its output to convey diagnostic or therapeutic information. For example, in various embodiments, information regarding the ECG signal can be conveyed using color and/or pattern effects on the display 110. In several embodiments, these color and pattern effects can indicate the signal's rhythm state and can be displayed in the background behind an ECG trace. In some embodiments, laypersons can use the color or pattern to recognize a healthy or treatable ECG rhythm state, and clinicians can use the color or pattern to aid rhythm diagnosis and therapy. In the case of a defibrillator, different colors/patterns can be used to quickly indicate the presence of specific shockable rhythms (such as ventricular fibrillation or ventricular tachycardia) and specific non-shockable rhythms (such as asystole or sinus rhythm). The dynamic and time-varying nature of the color/pattern can also assist in assessing a broader view of the heart's health and current state, as the history of the rhythm changes can convey important clinical information about the current and future state of the heart.

While both background colors and patterns can be used to quickly convey diagnostic information to a user, each schema can offer particular advantages and can be employed in various forms. Referring first to color schemas, the use of background color to convey information about a real-time or static ECG trace can be implemented by dynamically changing the color underlying the ECG trace on the display 110 to indicate the rhythm state. The actual rhythm state itself can be determined by the rhythm analysis algorithms performed by the processor 120. The specific colors and corresponding rhythm types can be selected to suit the user's needs, and to match what the underlying analysis algorithms are able to diagnose. Different schemas can be used depending on the application. For instance, a “Cardiac Arrest Mode” may identify rhythm types relevant to the arrest and have only categories relevant to that specific situation (e.g., “shockable” vs. “non-shockable”), while a “Diagnostic Mode” may have the ability to indicate more specific arrhythmias. One embodiment of background color-coding for use in a defibrillator or AED, or for reviewing an ECG trace of a cardiac arrest, is summarized below.

A) Green for Organized rhythms capable of producing a pulsatile blood flow
1. Bright green for rhythms with rates from 50 to 150
2. Light green for Bradycardia rhythms with rates from 10 to 50
3. A different shade of green or a pattern can indicate if pharmacologic therapy would be useful to the patient

B) Yellow for Asystole (e.g., the absence of rhythm and not capable of supporting blood flow and perfusion)

C) Red for rhythms requiring electrical shock
1. Bright red for Ventricular Fibrillation (VF)
2. Bright orange-red for Ventricular Tachycardia
3. Light orange for low amplitude VF
4. A different shade of red or a pattern can indicate which VF episodes should be shocked directly and which should receive Cardiopulmonary Resuscitation (CPR) chest compressions first

D) Blue for artifacts that obscure the signal
1. Defibrillatory shocks
2. Disconnected pads

The colors and associated descriptions in the above schema are merely representative of a particular embodiment of the technology, and can be varied in further embodiments. In various embodiments, any color can be chosen for a particular rhythm state. Further, the colors can be selected based on ranges most useful to the user. For example, while the above schema designates different green hues for organized rhythms greater or less than 50 beats per minute, such a cut-off can be altered to best serve the user. In some embodiments, a particular color/diagnosis correlation can be selected based on color associations (e.g., green is generally seen as ‘good’, while red is usually seen as ‘bad’). Additionally, in various embodiments, the color may or may not be overlaid with the ECG signal.

Referring now to pattern schemas, to emphasize the display, and enable color-blind users to use the method effectively, patterns may additionally or alternately be used. When used in conjunction with color schemas, the presence of patterns allows for a representation of a greater number of rhythm states. For instance, in the “Cardiac Arrest Mode” described above, a red background behind the ECG trace may indicate VF that is not yet ready to be shocked (and is in need of more CPR), and a red background with flashing diagonal lines may indicate that the VF is primed for defibrillation. In this manner, patterns may facilitate the directions for therapy and care, especially in stressful resuscitation situations. In one example, using current and future predictive analysis algorithms, different types of VF could be indicated based on the probability that the VF will convert to a sinus rhythm when shocked. It is possible that given the same rhythm diagnosis, the recommended treatment may be different depending on other patient characteristics, and the use of different pattern or color combinations can facilitate a way to quickly communicate this information to the clinician. Various embodiments can use static and/or dynamic (e.g., flashing) patterns. Static patterns can include horizontal stripes of contrasting shades of color, vertical bands of contrasting color, diagonal stripes of color, checker board patterns, or other textures of contrasting colors to be developed for different indications.

Flashing patterns can include alternating changes in color which “flash” to indicate the immediate need for a change in care. For instance, in some embodiments, when VF occurs and immediate shock is important, the diagonal stripes can alternate in a red/white background color pattern so that each stripe can change from red to white and back to red every time interval (e.g., each second). In some embodiments, this method of notification can be reinforced with an audible alert (e.g., an alarm). In some embodiments, the tempo of the alternating flashing stripes could be increased as the defibrillator charges to give an alert to the rescuing team that a shock is about to be delivered.

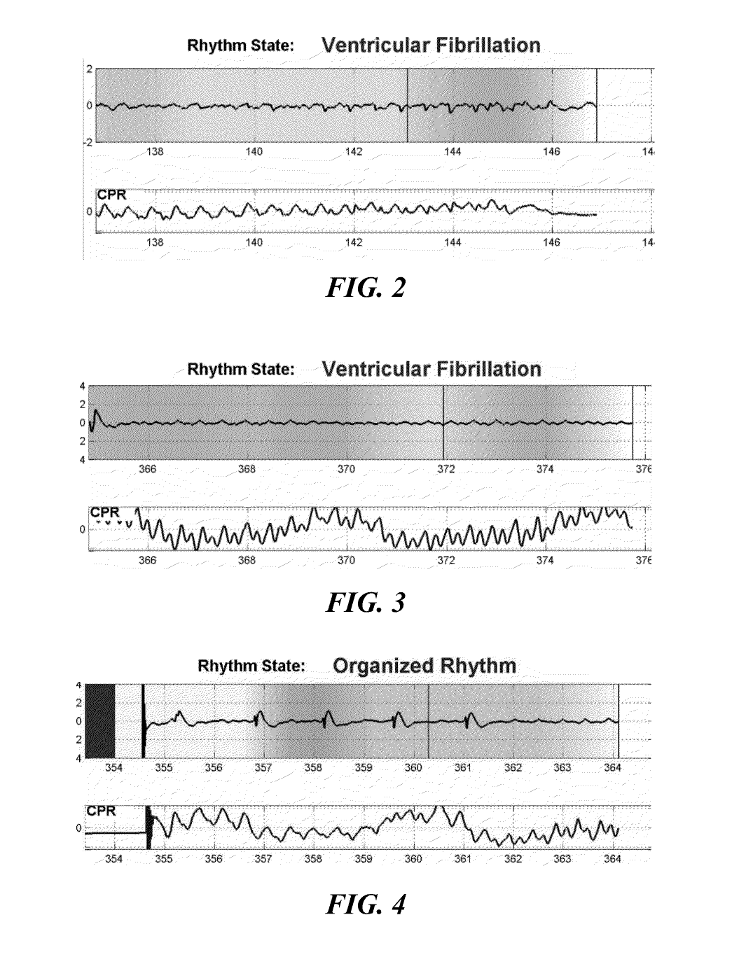

As discussed above, the color/pattern schemas can be used in real time with a continuously-updating trace, or as a case review analysis post-event. In embodiments utilizing real-time analysis, the color/pattern on the display 110 can update as the rhythm is diagnosed, and as the ECG is produced by the patient. FIGS. 2-4 illustrate real-time background color changes as the ECG is being displayed. FIGS. 2-4 each include a top pane illustrating the ECG, with the x-axis in seconds and the y-axis in milliVolts, and a bottom pane illustrating the chest's electrical impedance. The bottom pane can be used to indicate that chest compressions are occurring and that the ECG trace above is obscured by CPR chest compression artifacts and cannot be reliably read.

Referring first to FIG. 2, an ECG (the top trace) is illustrated with a red background for stronger VF and an orange background for finer VF (both potentially shockable rhythms with slightly different clinical implications). The analysis window is indicated by the vertical black lines. As described above, the bottom trace is the chest impedance, and can be used to help the user determine the presence of CPR or movement artifact in the case of a review. FIG. 3 illustrates an ECG with the end of a cardiac beat followed by VF. The transition from green (non-shockable rhythm) to red/orange can indicate onset of VF and can alert the clinician to the change in status. FIG. 4 illustrates a defibrillatory shock (blue) followed by an organized rhythm (green). In further embodiments, the color could pulsate or be hatched or striped to indicate that drug therapy could be considered.

In the illustrated embodiments, the background color is changed as the diagnostic window (e.g., 3.8 seconds in this case) is determined. The color slowly fades from one shade to another when the diagnosis changes, indicating how certain the algorithm is as to the type of rhythm present. In this manner, transients and artifacts can be overlooked in a qualitative manner by the observer, as they will be indicated by a short slight color tint change rather than a full steady color change. In the event that a “misread” were to occur, this would be a single subtle change in the shade of the continuously displayed background which would then revert to the correct color on the next read when the error is corrected. This would provide continuous feedback to the clinician team of the current state of the rhythm but would also allow an occasional misread to not communicate a false impression. These changes can allow a dynamic display of the rhythm reading at each 3.8 second interval such that the change in the cardiac rhythm would be immediate and obvious to the clinician. FIGS. 2-4 are merely representative of possible ECG readings and display schemas. Further embodiments can include any of the color/pattern display settings described above or other suitable colors/patterns.

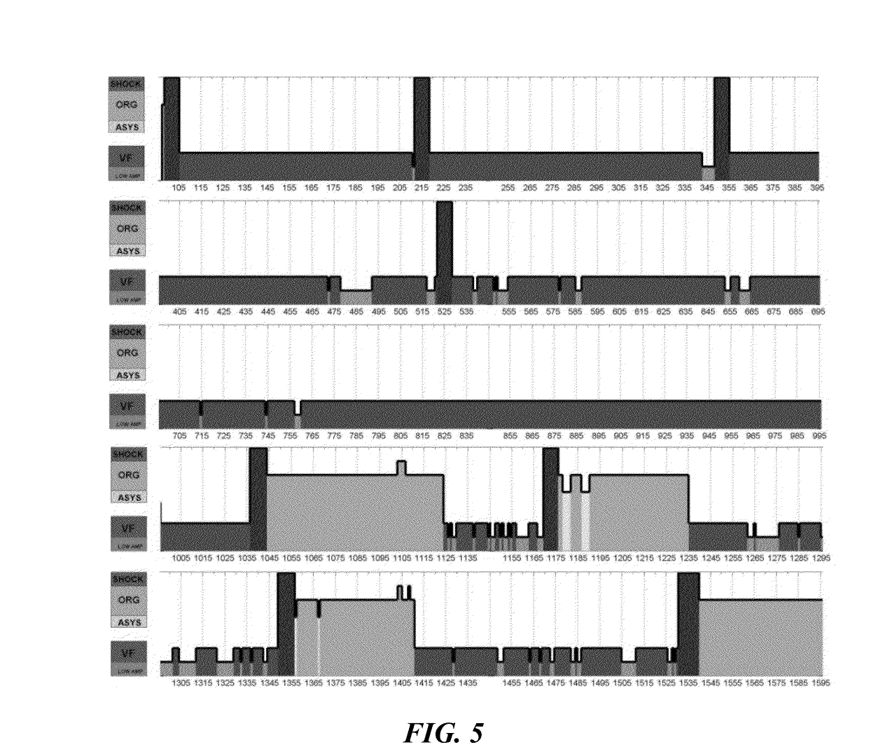

In addition to a moving, real-time display, an entire pre-recorded ECG case may be analyzed and annotated using a simplified color scheme. The ECG can be recorded either in the field or in the clinical setting. Some embodiments can include the ability to “zoom out” for a “bird's eye” view of the case to give a color-coded overview of the ECG trace. For example, in some embodiments, a significant length of the signal (e.g., 20 minutes) can be displayed on the screen, as compared to approximately 10-20 seconds in a real-time display. The ECG trace itself may or may not be superimposed over the background color/pattern. FIG. 5 illustrates a birds-eye case review of approximately 30 minutes of a color-coded rhythm diagnosis, without an ECG trace overlay. In this manner, the course of an extended period of time, such as an entire cardiac arrest, can be quickly and easily assessed. In FIG. 5, the times where the patient is in VF (red), shocked (blue), and returns to organized rhythm interspersed with asystole (green with some yellow) are easily identified and an overall summary of the arrest can be understood quickly and simply.

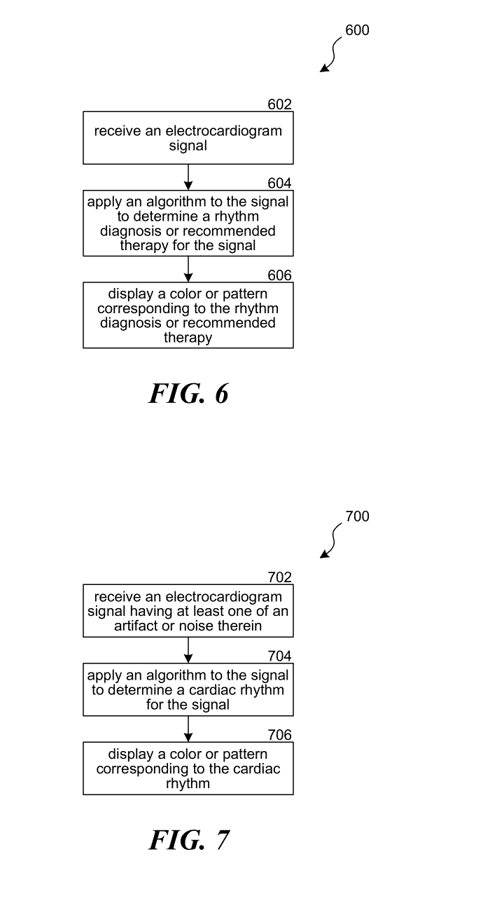

FIG. 6 is a block diagram illustrating a method 600 of displaying an ECG rhythm. At blocks 602 and 604, the method 600 includes receiving an ECG signal and applying an algorithm to the signal to determine a rhythm diagnosis or recommended therapy for the signal. In some embodiments, the rhythm is determined to be one of an organized rhythm, asystole, ventricular fibrillation, or ventricular tachycardia. In particular embodiments, the signal has signal artifacts or signal noise therein.

At block 606, the method 600 can include displaying a color or pattern corresponding to the rhythm diagnosis or recommended therapy. In some embodiments, the color or pattern can be displayed as a background to an overlaying display of the signal. In various embodiments, the color/pattern can comprise a static or flashing color or pattern on the display. In some embodiments, the color/pattern display is continuously updated to correspond to the rhythm diagnosis or recommended therapy. In various embodiments, the method 600 can be performed in real-time or can be performed on a pre-recorded signal. In some embodiments, the method 600 can further include initiating at least one of an audio or visual alarm in response to the determined rhythm diagnosis or recommended therapy.

FIG. 7 is a block diagram illustrating a method 700 of conveying information about an ECG rhythm. At block 702, the method 700 can include receiving an ECG signal with the ECG signal including at least one of artifacts or noise therein. In some embodiments, the signal has a visually indecipherable rhythm. At block 704, the method 700 can include applying an algorithm to the signal to determine a cardiac rhythm for the signal. At block 706, the method 700 can include displaying a color or pattern corresponding to the cardiac rhythm. In some embodiments, the displaying is continuously updated to correspond to the cardiac rhythm.

The systems and methods described herein offer several advantages over currently available methods of monitoring cardiac rhythms. For example, the method of color- and/or pattern-coding the background of the ECG trace can improve patient care as well as facilitate easier review of ECGs. Quick analysis and diagnostic recommendations enabled by the present technology can improve patient outcomes and reduce error. In cases where the ECG trace is obscured by artifact and is thus unusable to a clinician, but the underlying analysis algorithms are still able to accurately diagnose the signal, a clinician may rely on the color scheme (rather than simply a text display) to help interpret the rhythm and decide on the appropriate therapy. In lieu of a visually decipherable ECG, a recent history (e.g., approximately 20 seconds) of the ECG state can be preserved by means of the color changes, rather than requiring a list of text annotations to indicate the most recent rhythm state transitions.

From the foregoing it will be appreciated that, although specific embodiments of the technology have been described herein for purposes of illustration, various modifications may be made without deviating from the spirit and scope of the technology. Further, certain aspects of the new technology described in the context of particular embodiments may be combined or eliminated in other embodiments. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/we claim:

1. A method of displaying an electrocardiogram rhythm, the method comprising:
receiving an electrocardiogram signal;
applying an algorithm to the electrocardiogram signal to determine a rhythm diagnosis or recommended therapy; and
displaying a color and/or a pattern corresponding to the rhythm diagnosis or recommended therapy.

2. The method of claim 1 wherein displaying a color and/or a pattern corresponding to the rhythm diagnosis or recommended therapy comprises displaying the color and/or pattern as a background to an overlaying display of the signal.

3. The method of claim 1 wherein displaying a color and/or a pattern corresponding to the rhythm diagnosis or recommended therapy comprises continuously updating the displaying to correspond to the rhythm diagnosis or recommended therapy.

4. The method of claim 1, further comprising initiating at least one of an audio or visual alarm in response to the determined rhythm diagnosis or recommended therapy.

5. The method of claim 1 wherein displaying a color and/or a pattern corresponding to the rhythm diagnosis or recommended therapy comprises flashing the color and/or pattern.

6. The method of claim 1 wherein receiving the electrocardiogram signal comprises recording the signal, and wherein displaying a color and/or a pattern corresponding to the rhythm diagnosis or recommended therapy comprises annotating the recorded signal with a color and/or a pattern corresponding to the rhythm diagnosis.

7. The method of claim 1 wherein applying the algorithm and displaying the color and/or pattern are performed in real-time along with receiving the electrocardiogram signal.

8. The method of claim 1 wherein applying the algorithm to the electrocardiogram signal to determine the rhythm diagnosis comprises determining if the signal indicates one of an organized rhythm, asystole, ventricular fibrillation, ventricular tachycardia, or other cardiac physiological state.

9. The method of claim 1 wherein receiving the electrocardiogram signal comprises receiving a signal having signal artifacts or signal noise therein.

10. The method of claim 1 wherein displaying the color and/or a pattern comprises simultaneously displaying a color and/or a pattern corresponding to the rhythm diagnosis or recommended therapy.

11. An electrocardiography system, comprising:
a display device; and
a physical computer-readable storage medium having stored thereon, computer-executable instructions that, if executed by a computing system, cause the computing system to perform operations comprising
receiving an electrocardiogram signal;
determining a rhythm diagnosis or recommended therapy for the signal; and
transmitting instructions to the display device to display at least one of a color or a pattern corresponding to the rhythm diagnosis or recommended therapy.

12. The electrocardiography system of claim 11 wherein transmitting instructions to the display device to display the color or pattern corresponding to the rhythm diagnosis or recommended therapy comprises continuously transmitting instructions to the display device to display at least one of the color or pattern as a background to an overlaying display of the signal.

13. The electrocardiography system of claim 11 wherein the display device comprises at least one of an electrocardiogram monitor, a defibrillator, or an automated external defibrillator.

14. The electrocardiography system of claim 11, further comprising an audio alarm, wherein the operations further comprise directing the alarm to activate in response to a determined rhythm diagnosis.

15. A method of conveying information about an electrocardiogram rhythm on a visual display, the method comprising:
receiving an electrocardiogram signal from a human patient, the electrocardiogram signal having at least one of artifacts or noise therein;
processing the electrocardiogram signal via an algorithm to determine a cardiac rhythm for the signal; and
displaying a color and/or a pattern corresponding to the cardiac rhythm on the visual display.

16. The method of claim 15 wherein receiving the electrocardiogram signal comprises receiving an electrocardiogram signal having a visually indecipherable rhythm.

17. The method of claim 15 wherein the displaying is performed in real time with the receiving.

18. The method of claim 15 wherein displaying the color and/or the pattern corresponding to the cardiac rhythm comprises displaying the color and/or pattern as a background to an overlaying display of the signal on the visual display.

19. The method of claim 15 wherein displaying the color and/or the pattern corresponding to the cardiac rhythm comprises continuously updating the displaying to correspond to the cardiac rhythm.

20. The method of claim 15 wherein displaying the color and/or the pattern corresponding to the cardiac rhythm comprises displaying at least one of horizontal stripes of contrasting shades of color, vertical bands of contrasting color, diagonal stripes of color, or a checkerboard pattern.
